(19) United States
(12) Patent Application Publication
(10) Pub. No.: US 2019/0189275 A1
(43) Pub. Date: Jun. 20, 2019

(54) First Trainable Robot Doctor Named AIPD Using New Methods and Systems for Its Artificial Brain

(71) Applicants: Gewei Ye, Timonium, MD (US); Jessica Zhu Ye, Timonium, MD (US)

(72) Inventors: Gewei Ye, Timonium, MD (US); Jessica Zhu Ye, Timonium, MD (US)

(21) Appl. No.: 15/844,666

(22) Filed: Dec. 18, 2017

(51) Int. Cl. G16H 50/20; G06N 3/08; G06F 17/28

(52) U.S. Cl. CPC G16H 50/20; G06F 17/28; G06N 3/08

(57) ABSTRACT

Only a small percentage of our population (e.g., the rich and power) can afford dedicated personal human doctors hence live healthier and longer. Healthcare inequality is a fundamental challenge to our society. Social and policy solutions such as ObamaCare are difficult to implement. Our invention intends to provide a technology solution by getting everyone a data-driven artificial intelligence (AI) personal doctor (AIPD), i.e. the first trainable Robot Doctor. We invent new methods and systems to create and train the artificial brains (AB) of the first trainable robot doctor named AIPD on NLP and deep learning technologies. The AB of AIPD can be pre-trained with validated health literature from authorities and can be continuously trained with NLP and deep learning algorithms by the growing literature and user contacts. The first robot doctor's response by its AB can be validated by a human doctor or can be cross-validated from another AIPD robot doctor with a different trainable artificial brain.

### BACKGROUND

### 1.1 FIELD OF THE INVENTION

Only a small percentage of our population (e.g., the rich and power) can afford dedicated personal human doctors hence live healthier and longer. Healthcare inequality is a fundamental challenge to our society. Social and policy solutions such as ObamaCare are difficult to implement. We invent the artificial intelligence (AI) solutions to get everyone a data-driven AI personal doctor (AIPD). i.e., the first trainable robot doctor.

### 1.2 DESCRIPTION OF THE RELATED ART

The artificial brain of the first trainable Robot Doctor named AIPD is designed and implemented in three phases: (1) release the mobile and Web portal on data-driven AI machines to connect lab tests to diet and weight loss solutions. The lab-tests section of the Portal focuses on answering “are my lab-tests numbers normal?” The diet section of the portal focuses on answering “how to improve the numbers and lose weight?” The weight-loss section of the portal tracks the weight-loss progress and focuses on answering “where is my BMI number in the BMI map:” (2) release the first trainable Robot Doctor named AIPD (need FDA certification—Dr. Scott Gottlieb. FDA Commissioner) that integrates with the mobile AIPD portal. One may want to try our pilot AIPD Robot Doctor in their infancy (sec FIG. 1) and will be helping teach the robot doctor to grow up with each contact; (3) release AIPD with the capability of human doctor validation (no need of FDA certification—Dr. Scott Gottlieb. FDA Commissioner). The “chat with a human doctor” window (see FIG. 2) enables the validations from human doctors on the AIPD's responses to patients' questions.

The new methods (i.e., the NLP methods of FIG. 4) and systems (i.e., the unique AIHPC and PNN algorithms of FIG. 5) for the unique trainable artificial brain (see FIG. 4 and FIG. 5) of (he first Robot Doctor named AIPD (see FIG. 3) are created by unique new ways of integrating the next-gen technologies (see FIG. 6) including IBM Watson. AIHPC®. Natural Language Processing, machine learning (i.e., PNN). speech recognition, and text to speech etc. AIHPC stands for Artificial Intelligence High Performance Computing (see http://veswici.com/ui/w/ai/). PNN stands for Predictive Neural Network (see http://veswici.com/ui/w/p/). AIHPC and PNN are new methods and systems that are documented in a separate USPTO patent application.

The unique differentiating features for the artificial brain of the first Robot Doctor named AIPD arc: (1) compared to human personal doctors. Robot Doctor AIPD is more accessible through mobile. Web, or other robot machines anytime (24×7) anywhere; (2) compared to general-purpose Siri like NLP apps. AIPD specializes in medical and health artificial intelligence with both natural language processing (e.g., by IBM Watson) and quantitative/diagnostic/predictive deep-learning (e.g., by AIHPC and PNN) capabilities for medical problems; (3) the NLP trained, adaptive, and validated knowledge base of the artificial brain: the artificial brain of AIPD is pre-trained with validated medical and health literature from authorities and can be continuously trained with deep learning NLP algorithms by the growing literature and user contacts. Traditional automated symptom checkers do not have the adaptive NLP trained AI machine powered by IBM Watson and AIHPC® (ref. comments from a colleague of Johns Hopkins Medical School); (4) the validation capability by human doctors or another robot doctor: the response by the artificial brain of AIPD may be validated by human doctors; the response can also be cross-validated by another AIPD with a different artificial brain.

### BRIEF DESCRIPTION OF THE DRAWINGS

FIG. 1—Mobile and Web Interface of the First Trainable Robot Doctor Named AIPD

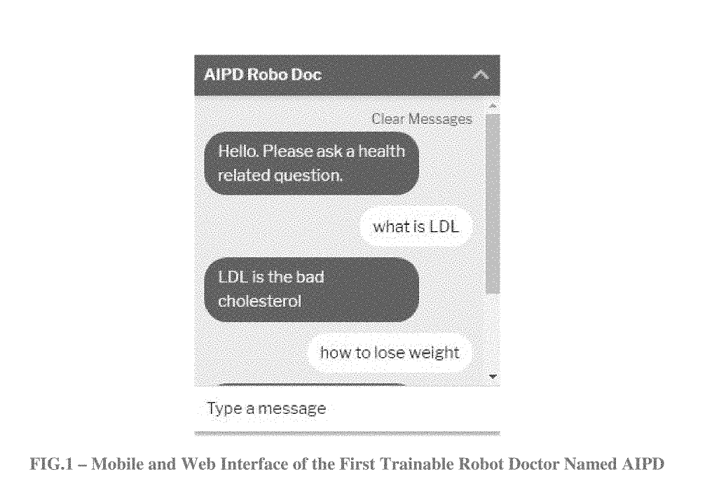

FIG. 2—Human Validation Interface for the First Trainable Robot Doctor AIPD

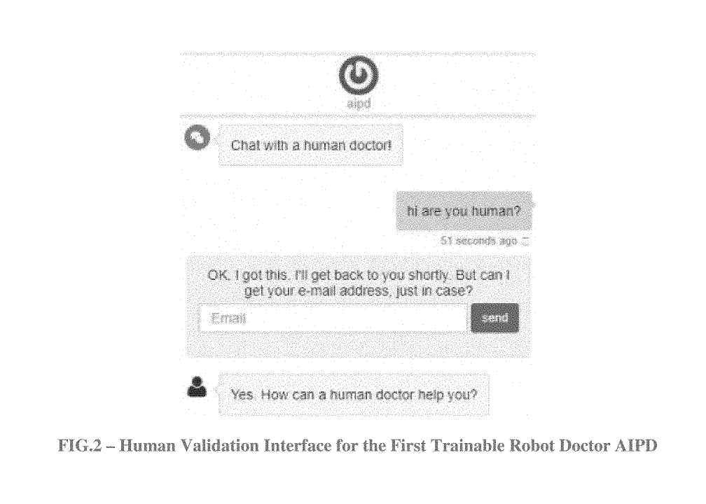

FIG. 3—New NLP Methods and AIHPC Systems for the First Robot Doctor AIPD

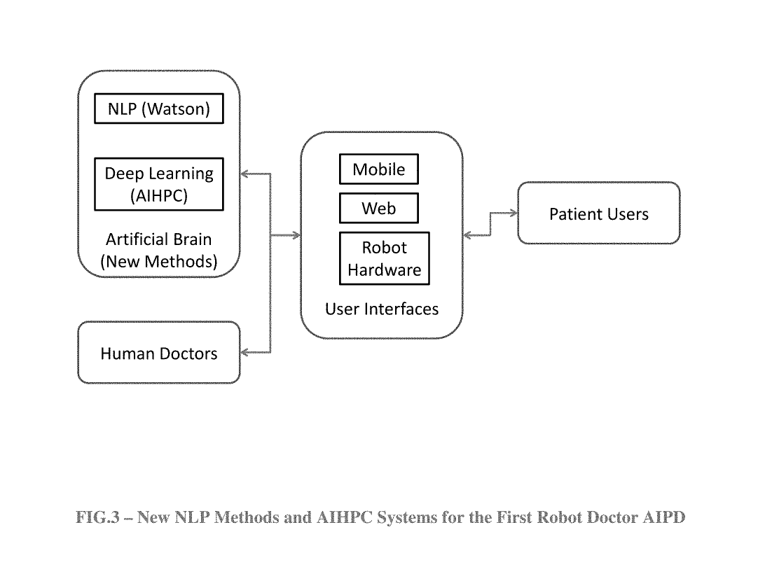

FIG. 4—New NLP Methods of the Artificial Brain of Robot Doctor AIPD on Weight Loss

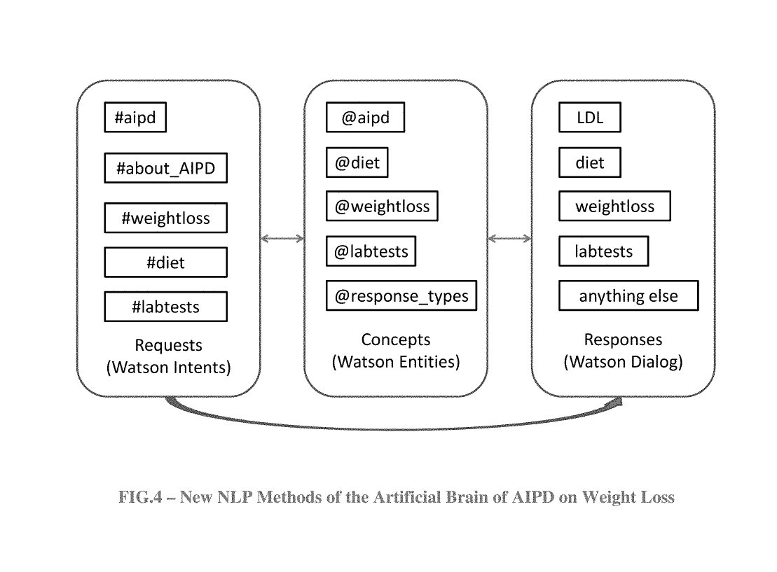

FIG. 5—Unique Data-driven Systems Design for the First AIPD Artificial Brain

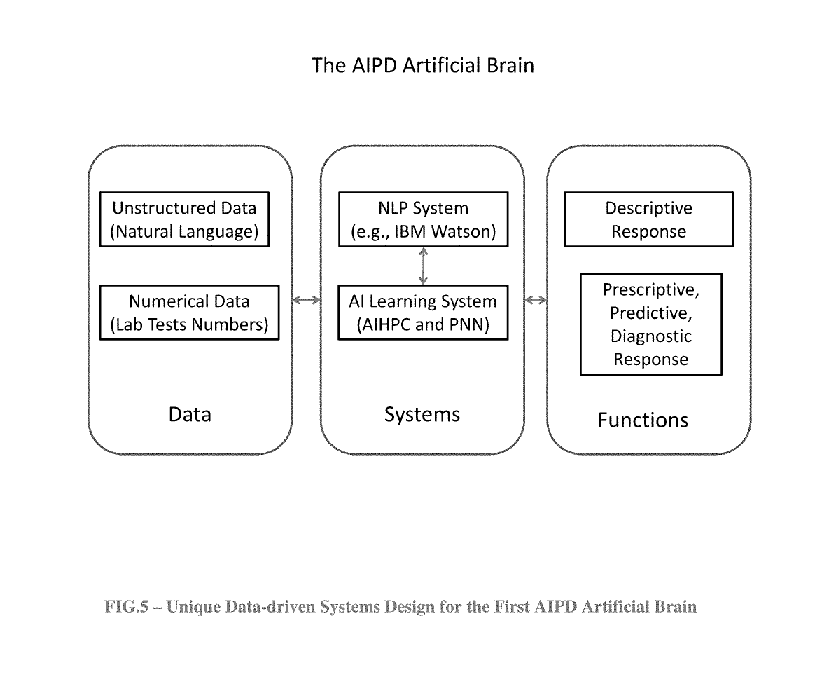

FIG. 6—Unique Systems Design for the First AIPD Hardware Robot

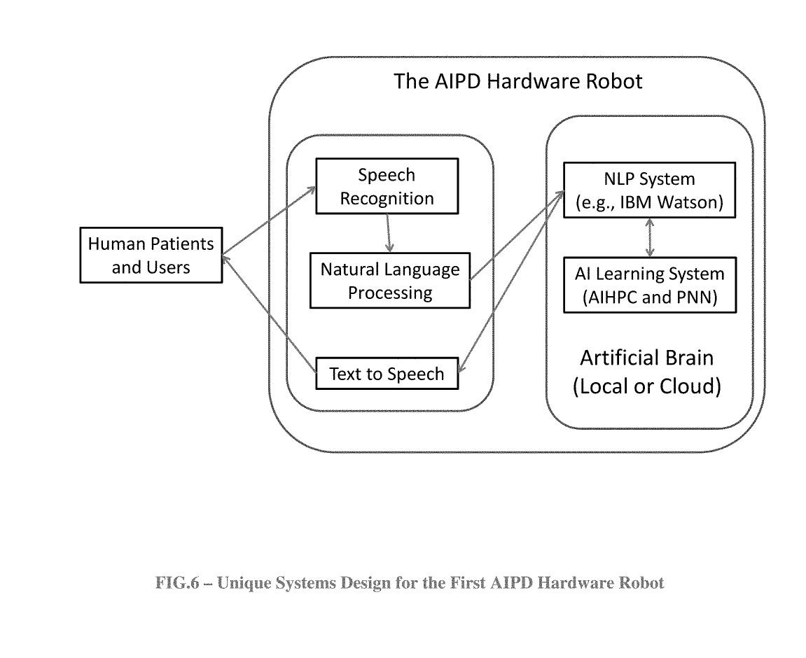

1. our invention includes the new methods and systems to create and train the first artificial brain of robot doctors with natural language processing (NLP) and deep learning technologies. These comprise: (1) the unique NLP components and algorithms to create and train the artificial brains that can understand and interpret users' medical questions in natural languages; (2) the unique deep learning (AIHPC and PNN) algorithms to train the artificial brains that analyze the users' medical questions and prepare intelligent responses; (3) the cross-validation capability of the trainable artificial brains by another AIPD artificial brain that may be automatically trained by user contacts or manually trained by enhancing the NLP and deep learning backend.

2. our invention includes the software implementations and unique systems designs of the new methods as the first trainable robot doctor systems (e.g., Robot Doctor ATPD or Robo Doc ATPD) that may be operated on Web, mobile devices, robot hardware, or other forms. Claim 2 comprises all types of the trainable robot doctors that automatically interact, analyze, and respond to patients' medical problems. These robot doctors may cover for all types of human medical doctors who are practicing weight loss, internal medicine, specialized medicine, pediatrics, cardiology, dermatology, pharmacy, lab tests, symptom checks, diagnostics, etc.

3. our invention includes the unique software implementation and systems designs of the new methods and systems as the trainable Robot Doctor AIPD to solve the weight-loss problem for public health. The artificial brain of the Robot Doctor AIPD on Weight Loss understands and analyzes users' medical questions and then intelligently provides adaptive and relevant responses on weight loss, diet, and lab tests. It is trainable manually and automatically and grows up with each of the user contacts.
